(19) United States
(12) Patent Application Publication
(10) Pub. No.: US 2015/0216613 A1
(43) Pub. Date: Aug. 6, 2015

(54) SKIN LANDMARK AND ANOMALY MEASUREMENT TOOL

(71) Applicant: Janel Parker Schilling, Portland, OR (US)

(72) Inventor: Janel Parker Schilling, Portland, OR (US)

(21) Appl. No.: 14/582,821

(22) Filed: Dec. 24, 2014

Related U.S. Application Data
(60) Provisional application No. 61/935,699, filed on Feb. 4, 2014.

(51) Int. Cl. A61B 19/00; A61B 5/00

(52) U.S. Cl. CPC A61B 19/46; A61B 5/4312

(57) ABSTRACT

Some embodiments of the present disclosure include a device for recording the location, size, and shape of anomalies and skin landmarks, such as scars, mass clusters, and the like. The device may include a substantially sector-shaped semitransparent marking base having a plurality of transparent regions and a removable backing. The marking base may include a plurality of semitransparent marking scales that define the transparent regions, wherein the entire device is configured to allow a user to trace anomalies and skin landmarks thereon, creating a permanent, accurate, and actual-size mapping of the subject's skin. In some embodiments, the marking base may be made of a vinyl material.

### RELATED APPLICATION

This application claims priority to Provisional Patent Application U.S. Ser. No. 61/935,699 filed on Feb. 4, 2014, the entire contents of which is herein incorporated by reference.

### BACKGROUND

The embodiments herein relate generally to medical devices, and more particularly, to a device for recording the location, size, and shape of anomalies and skin landmarks.

It is difficult to be consistently accurate with locating and recording the size and shape of anomalies on or deep to a patient's skin. Conventionally, for a patient's permanent record, an ultrasound technologist (sonographer) draws a much smaller version of what was seen onto a small drawing of a breast or other body part. The shape and location of each anomaly is estimated on this smaller than life size drawing.

Sometimes, there are multiple similar looking masses in the same area of the body part, and the technologist may confuse one mass with another. For follow-up exams, anomalies may be time consuming to find, and their exact location may be uncertain. This is particularly an issue for patients with large breasts and with patients who have undergone effective cancer therapy. If the therapy has caused the mass to disappear, the technologist cannot prove, or even be sure, that he or she is imaging the correct site of interest.

Therefore, what is needed is a device to record skin markings and skin landmarks and anomalies precisely, and in actual size, as a template.

### SUMMARY

Some embodiments of the present disclosure include a device for recording the location, size, and shape of anomalies and skin landmarks, such as scars, mass clusters, fluid collections deep to the skin, and the like. The device may include a substantially sector-shaped marking base having a plurality of transparent regions and a removable backing. The marking base may include a plurality of marking scales, which are also somewhat transparent, that define the transparent regions, wherein the entire tool is configured to allow a user to trace skin anomalies and landmarks thereon, creating a permanent, accurate, and actual-size mapping of the subject's skin. In some embodiments, the marking base may be made of a vinyl material.

### BRIEF DESCRIPTION OF THE FIGURES

The detailed description of some embodiments of the invention is made below with reference to the accompanying figures, wherein like numerals represent corresponding parts of the figures.

FIG. 1 is a perspective view of one embodiment of the present invention.

FIG. 2 is a perspective view of one embodiment of the present invention.

FIG. 3 is an exploded view of one embodiment of the present invention in use.

FIG. 4 is a top view of one embodiment of the present invention shown in use.

FIG. 5 is a top view of one embodiment of the present invention shown in use.

FIG. 6 is a top view of two exemplary embodiments of the present invention combined and in use.

FIG. 7 is a perspective view of one embodiment of the present invention.

### DETAILED DESCRIPTION OF CERTAIN EMBODIMENTS

In the following detailed description of the invention, numerous details, examples, and embodiments of the invention are described. However, it will be clear and apparent to one skilled in the art that the invention is not limited to the embodiments set forth and that the invention can be adapted for any of several applications.

The device of the present disclosure may be used to record the location, size, and shape of anomalies and skin landmarks and may comprise the following elements. This list of possible constituent elements is intended to be exemplary only, and it is not intended that this list be used to limit the device of the present application to just these elements. Persons having ordinary skill in the art relevant to the present disclosure may understand there to be equivalent elements that may be substituted within the present disclosure without changing the essential function or operation of the device.

1. Marking Base

2. Backing

3. Transparent Regions

4. Marking Scales

The various elements of the device for recording the location, size, and shape of anomalies and skin landmarks of the present disclosure may be related in the following exemplary fashion. It is not intended to limit the scope or nature of the relationships between the various elements and the following examples are presented as illustrative examples only.

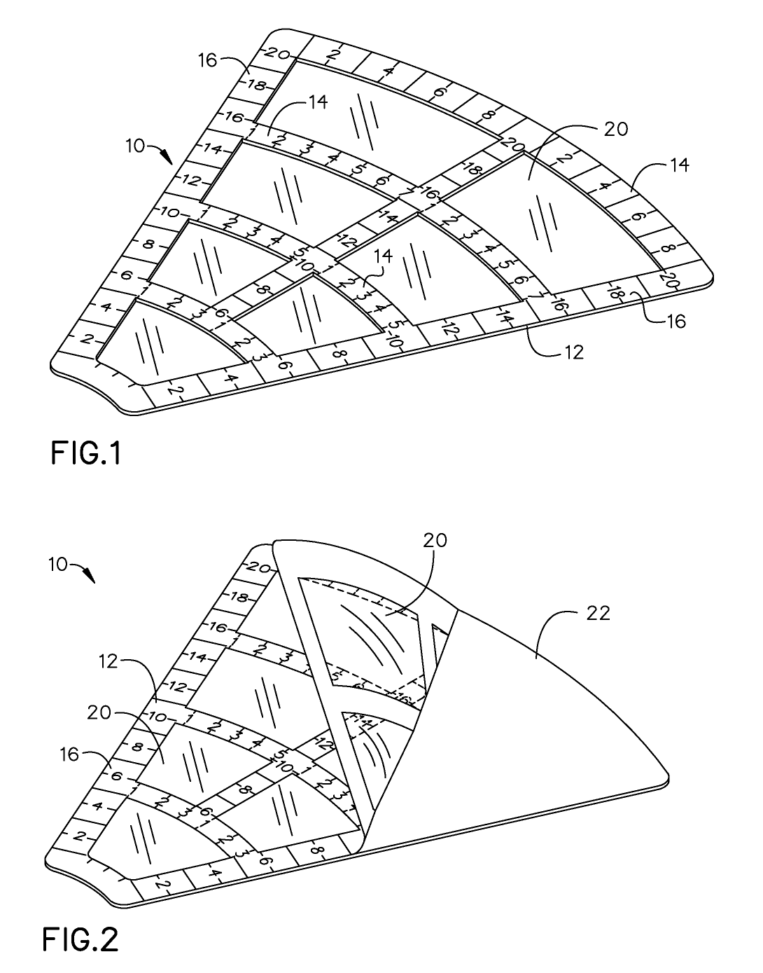

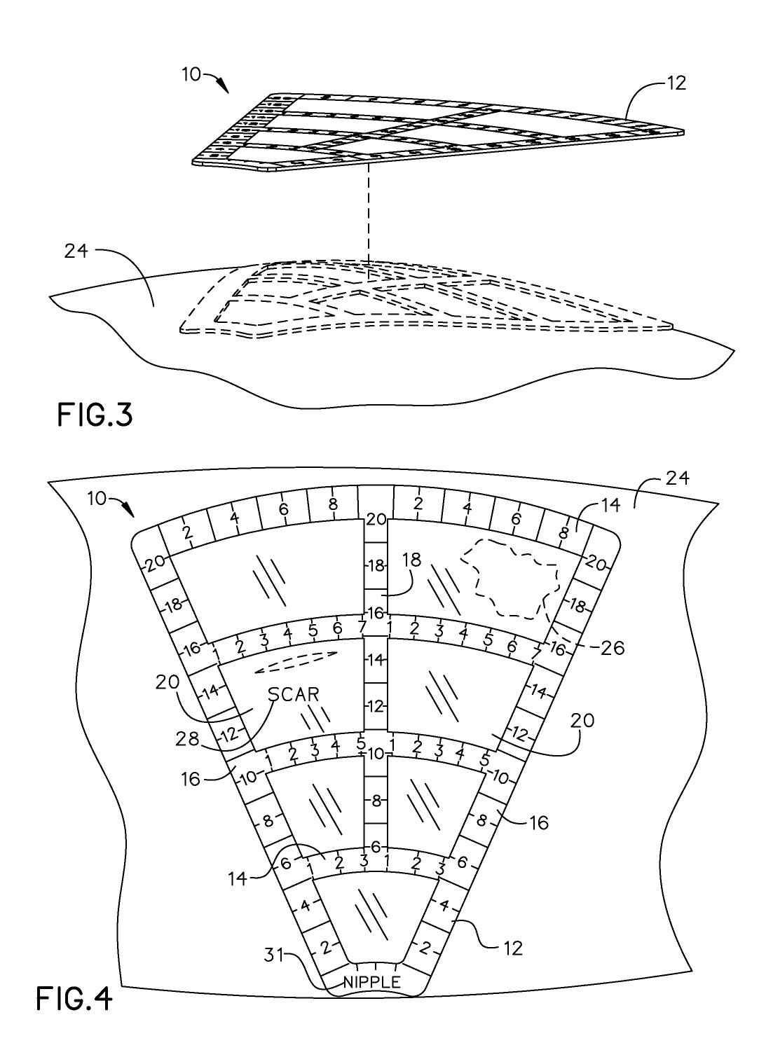

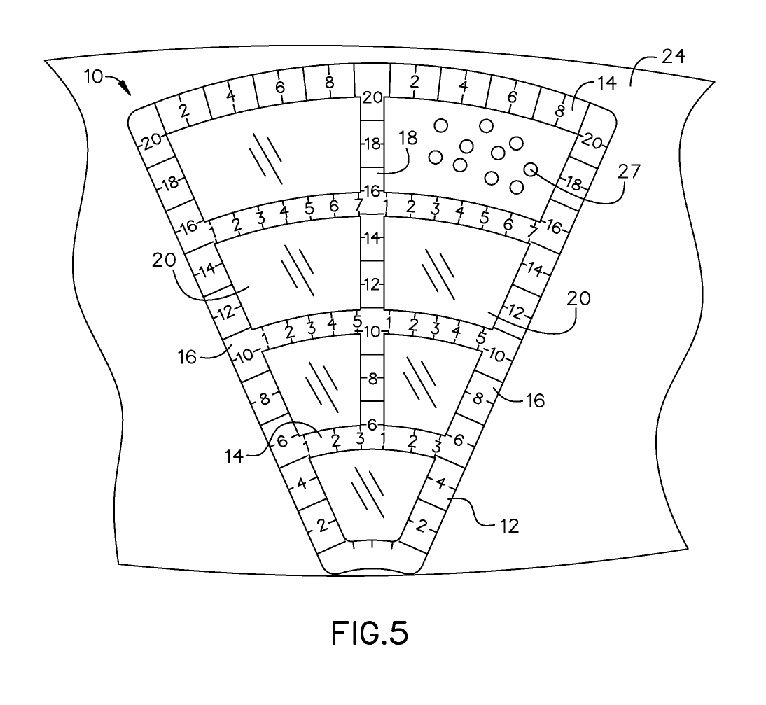

By way of example, and referring to FIGS. 1-7, some embodiments of the device 10 for recording the location, size, and shape of anomalies and skin landmarks of the present disclosure comprise a marking base 12 having a removable backing 22, wherein the marking base 12 comprises at least one transparent region 20 and a plurality of marking scales, such as a pair of vertical side marking scales 16, a plurality of radial marking scales 14, and a center marking scale 18, wherein the device 10 is configured to be applied by the user to a subject's skin 24 and the marked scale region and the transparent regions 20 allow a user to trace scars 28, landmarks 26, mass clusters 27 or the like directly on the marking base 12, creating a permanent, accurate, and actual-size record or mapping of a subject's skin and anatomy 24. For example, the device 10 may be used to map a breast, wherein breast markings 31 may also be noted on the marking base 12 to ensure that the device 10 can be accurately placed on the breast during follow-up appointments to locate anomalies and for comparison purposes, resulting in faster and more accurate follow-up exams.

As shown in the figures, the device 10 may be substantially sector-shaped, wherein the outer edge of the device 10 is defined by the vertical side marking scales 16 and two outer radial marking scales 14, wherein the marking scales may comprise distance markings similar to a ruler. For example, the marking scales may comprise centimeter markings In other embodiments, the marking scales may not comprise distance markings, but rather only create a grid for use by a technologist. The marking scales may be printed on to the marking base 12 using any suitable printing method. Additional radial marking scales 14 may be positioned between the outer radial marking scales 14 and may extend from a first of the vertical side marking scales 16 to a second of the vertical side marking scales 16. For example, in some embodiments, the device comprises about 5 radial marking scales extending from a first of the vertical marking scales to a second of the vertical marking scales, the radial marking scales being evenly spaced along a length of the vertical marking scales. In embodiments, the device 10 also comprises a center marking scale 18 extending from the largest radial marking scale 14 down the center of the device 10 towards the smallest radial marking scale 14, wherein the center marking scale 18 may be positioned between the pair of vertical marking scales 16 and may intersect a plurality of radial marking scales 14, creating a plurality of transparent regions 20. For example, in some embodiments, about 7 transparent regions 20 may be formed by the intersection of the marking scales 16, 14, and 18. In other embodiments, the entire marking base 12 may be substantially transparent.

The backing 22 may be initially removably attached to the marking base 12 and may be removed before use of the marking base 12, as shown in FIG. 2. Thus, the backing 22 may help keep the marking base 12 clean and free of debris prior to use thereof.

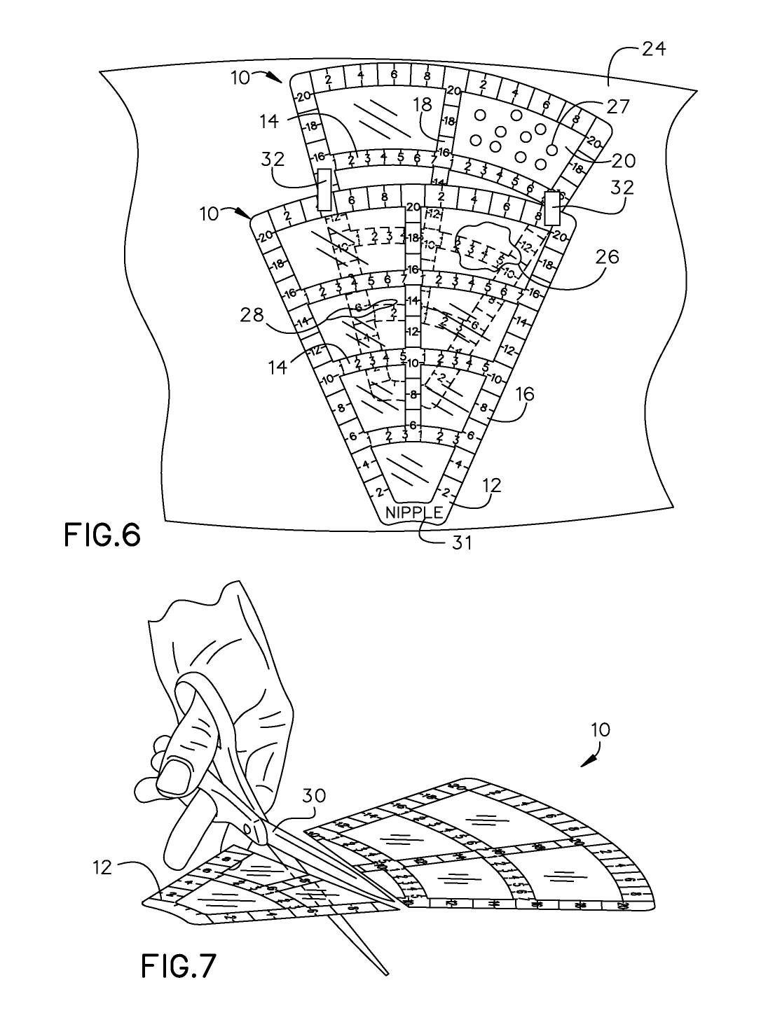

In embodiments, more than one device 10 may be used together, as shown in FIG. 6, when the area to be marked is quite large. In such an instance, the devices 10 may be attached using tape 32 or any other conventional fastener. Alternatively, the device 10 may be cut to a desired size and shape using, for example, scissors 30, as shown in FIG. 7.

The device 10 may be made from any suitable material and, in some embodiments, is pliable and conforms to the shape of the skin 24 to which it is applied. For example, the device 10 may be transparent, soft, and pliable. In some embodiments, the device 10 may be made of a vinyl material, such as 7 mil clear soft vinyl.

The device 10 may have any desired shape or size. In some embodiments, as mentioned above, the device 10 may be substantially sector-shaped, having a largest width of about 10 cm and a length of about 21 cm.

In addition to being used for mapping an area of skin 24, the device may have other uses, such as a tool for a seamstress to prevent the seamstress from making physical markings on her material.

Persons of ordinary skill in the art may appreciate that numerous design configurations may be possible to enjoy the functional benefits of the inventive systems. Thus, given the wide variety of configurations and arrangements of embodiments of the present invention the scope of the invention is reflected by the breadth of the claims below rather than narrowed by the embodiments described above.

What is claimed is:

1. A device for recording the location, size, and shape of anomalies and skin landmarks, the device comprising:
a marking base comprising at least one transparent region; and
a removable backing attached to the marking base, the removable backing configured to be removed, exposing a surface of the marking base that is to be applied to a member selected from the group consisting of a subject's skin and body part,
wherein:
the marking base comprises at least one marking scale, and
the at least one transparent region is configured to allow a user to trace skin anomalies and landmarks thereon, creating a permanent, accurate, and actual-size mapping of the user's skin.

2. The device of claim 1, wherein the marking base comprises:
a pair of vertical side marking scales;
a plurality of radial marking scales connecting the pair of vertical side marking scales; and
a center marking scale intersecting the radial marking scales and positioned between the vertical side marking scales.

3. The device of claim 2, wherein each of the marking scales comprises centimeter measurement marks.

4. The device of claim 2, wherein the intersections of the marking scales defines a plurality of transparent regions.

5. The device of claim 4, wherein the marking base comprises about 7 transparent regions.

6. The device of claim 1, wherein the entire marking base is substantially transparent.

7. The device of claim 1, wherein the device is substantially sector shaped.

8. The device of claim 7, wherein the device has a largest width of about 10 cm and a length of about 21 cm.

9. A device for recording the location, size, and shape of anomalies and skin landmarks, the device comprising:
a substantially sector-shaped marking base comprising a plurality of transparent regions; and
a removable backing attached to the marking base, the removable backing configured to be removed, exposing a surface of the marking base that is to be applied to a subject's skin,
wherein:
the marking base comprises a plurality of marking scales that define the transparent regions;
the transparent regions and semitransparent marking scales are configured to allow a user to trace skin anomalies and landmarks thereon, creating a permanent, accurate, and actual-size mapping of the subject's skin; and
the marking base comprises a vinyl material.

10. The device of claim 9, wherein the marking base comprises:
a pair of vertical marking scales;
about 5 radial marking scales extending from a first of the vertical marking scales to a second of the vertical marking scales, the radial marking scales being evenly spaced along a length of the vertical marking scales;
a center marking scale intersecting the radial marking scales and positioned between the vertical side marking scales; and
about 7 transparent regions defined by intersections of the semitransparent marking scales.
